(19) United States
(12) Patent Application Publication
(10) Pub. No.: US 2013/0090631 A1
(43) Pub. Date: Apr. 11, 2013

(54) MEDICAL DEVICE DELIVERY APPARATUS

(71) Applicant: BOSTON SCIENTIFIC SCIMED, INC., Maple Grove, MN (US)

(72) Inventors: James M. Anderson, Fridley, MN (US); Huisun Wang, Maple Grove, MN (US)

(73) Assignee: BOSTON SCIENTIFIC SCIMED, INC., Maple Grove, MN (US)

(21) Appl. No.: 13/649,250

(22) Filed: Oct. 11, 2012

Related U.S. Application Data
(60) Provisional application No. 61/545,775, filed on Oct. 11, 2011.

(51) Int. Cl. A61M 25/00; A61M 25/09

(52) U.S. Cl. 604/524

(57) ABSTRACT

The disclosure pertains to apparatus for intravascular delivery of medical devices past a juncture which requires an abrupt change of direction and methods of use therefor. The apparatus employs a directing element which deflects the medical device from an initial path to a second path. In some embodiments, the apparatus comprises more than one directing element and may be capable of redirecting by deflection more than one medical device.

### BACKGROUND

Intravascular devices, such as an embolic protection filter, are typically placed in a vessel such as an artery or vein to filter emboli contained in the blood stream. Examples of procedures employing such filters include angioplasty, atherectomy, thrombectomy, and stenting. These procedures generally involve transluminally inserting and delivering within the artery or vein an elongated wire and filter to a location distal a lesion. Once placed, a therapeutic device such as an angioplasty catheter is advanced along the wire to the site of the lesion to perform a therapeutic procedure (e.g. percutaneous transluminal coronary angioplasty, valve replacement, and the like.)

Certain intravascular procedures require that the guidewire and/or other intravascular device be advanced past a juncture within the vasculature at which the desired path requires an abrupt change of direction which is difficult to traverse with conventional guidewire systems. Attempts to address this difficulty with angled or steerable catheters or guidewires have been only partially successful. Accordingly, it is desirable to provide an improved system for directing guidewires and other medical devices past such junctures.

### SUMMARY

This disclosure pertains to a medical device delivery apparatus comprising an elongated support member having a proximal end, a distal end, and a longitudinal axis; a generally tubular element having at least a first lumen therethrough disposed at the distal end of the elongated support member; and a first directing element disposed at least partially within the generally tubular element and slidable within the first lumen thereof, wherein the first directing element has a first configuration which is generally straight and contained within the first lumen of the generally tubular element and a second configuration in which the first directing element is curved and at least partially extended distal of the generally tubular element, further wherein the first directing element is adapted to receive and redirect a first medical device advanced along a first path generally parallel to the longitudinal axis of the elongated support member proximate the distal end thereof and thereby direct at least a portion of the medical device along a second path forming a first internal angle of less than 120 degrees with the longitudinal axis when the first directing element is in the second configuration and the first medical device is extended distal of the generally tubular element.

In some embodiments, the disclosure pertains to a medical device delivery apparatus comprising two or more directing element adapted to receive and redirect a first medical device advanced along a first path generally parallel to the longitudinal axis of the elongated support member proximate the distal end thereof and thereby to direct at least a portion of the first medical device along a second path forming a first internal angle of less than 120 degrees with the longitudinal axis when the first directing element is in a second configuration and the first medical device is extended distal of the generally tubular element and a second directing element disposed at least partially within a second lumen of the generally tubular element and slidable within the second lumen thereof wherein the second directing element has a first configuration which is generally straight and contained within the second lumen of the generally tubular element and a second configuration in which the second directing element is curved and at least partially extended distal of the generally tubular element, further wherein the second directing element is adapted to receive and redirect a second medical device advanced along a path generally parallel to the longitudinal axis of the elongated support member proximate the distal end thereof and thereby direct at least a portion of the second medical device along a second path forming a first internal angle of less than 120 degrees with the longitudinal axis when the second directing element is in the second configuration and the second medical device is extended from of the generally tubular element.

In another embodiment, the disclosure relates to a method of positioning a medical device intravascularly comprising advancing a medical device delivery apparatus of any of claims 1-31 to a point within the vasculature; advancing a directing element from a first configuration in which the directing element is generally parallel to a longitudinal axis of a generally tubular element of the medical device delivery apparatus to a position in which at least a portion of the directing element is distal of the generally tubular element of the medical device delivery apparatus; causing the directing element to assume a second configuration in which at least a portion of the directing element is curved relative to the longitudinal axis of the generally tubular element of the medical device delivery apparatus; and advancing a medical device relative to the medical device delivery apparatus until the medical device contacts and is deflected from a first path generally parallel to the longitudinal axis of the generally tubular element of the medical device delivery apparatus to a second path forming an internal angle of less than 120 degrees with the first path.

### BRIEF DESCRIPTION OF DRAWINGS

FIGS. 1A-1D illustrate schematically the operation of the disclosed delivery device.

FIG. 2 illustrates an embodiment of a directing element of the disclosure.

FIGS. 3-3C illustrate portions of an embodiment of a medical device delivery apparatus of the disclosure.

FIG. 4 illustrates the distal region of an embodiment of a medical device delivery apparatus of the disclosure.

FIG. 5 is a view of the distal end of an embodiment of a medical device delivery apparatus of the disclosure.

FIG. 6 illustrates an alternate embodiment of a directing element of the disclosure.

FIG. 7 illustrates the distal region of an alternate embodiment of a medical device delivery apparatus of the disclosure.

FIG. 8 illustrates another alternate embodiment of a directing element of the disclosure.

FIG. 9 illustrates the distal region of an alternate embodiment of a medical device delivery apparatus of the disclosure.

FIGS. 10 and 11 illustrate the distal region of alternate embodiments of a medical device delivery apparatus of the disclosure.

FIGS. 12 and 13 illustrate the distal region of alternate embodiments of a medical device delivery apparatus of the disclosure which is adapted to deliver and deflect two medical devices by employing two deflection elements.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but not every embodiment may necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments, whether or not explicitly described, unless clearly stated to the contrary.

As used herein, the term “curved” should be interpreted as including both continuous smooth curves which are not necessarily arcs of circles as well as one or more generally straight segments joined by continuous smooth curves which are not necessarily arcs of circles. For example, two generally straight segments joined by a segment subtending a 135 degree arc of a circle should be considered to be encompassed by the term “curved” as applied to a “curved” component. Further, as applied to the angle between two joined paths or lines, the term “internal angle” should be interpreted as indicating the angle therebetween which is not greater than or equal to 180 degrees.

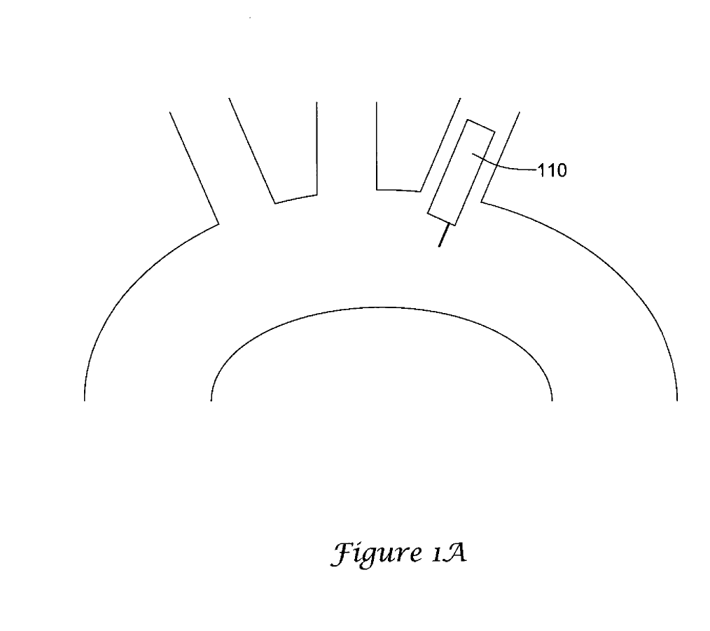

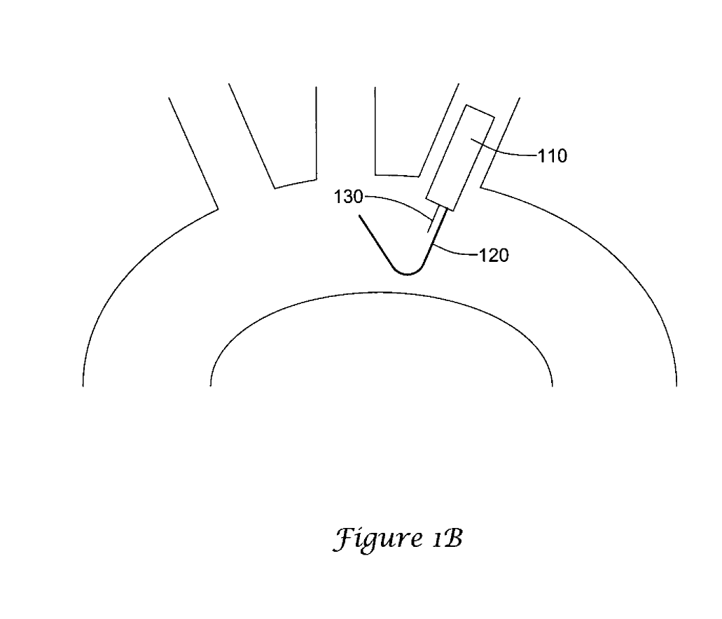

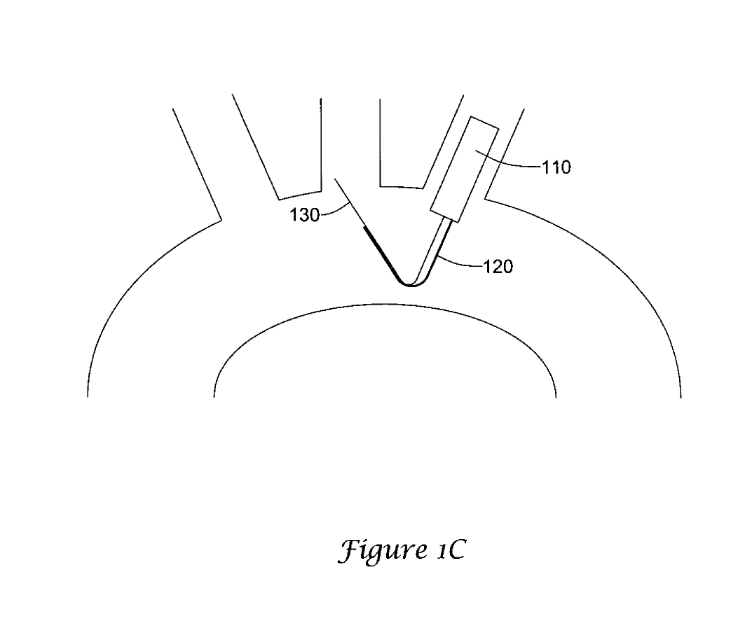

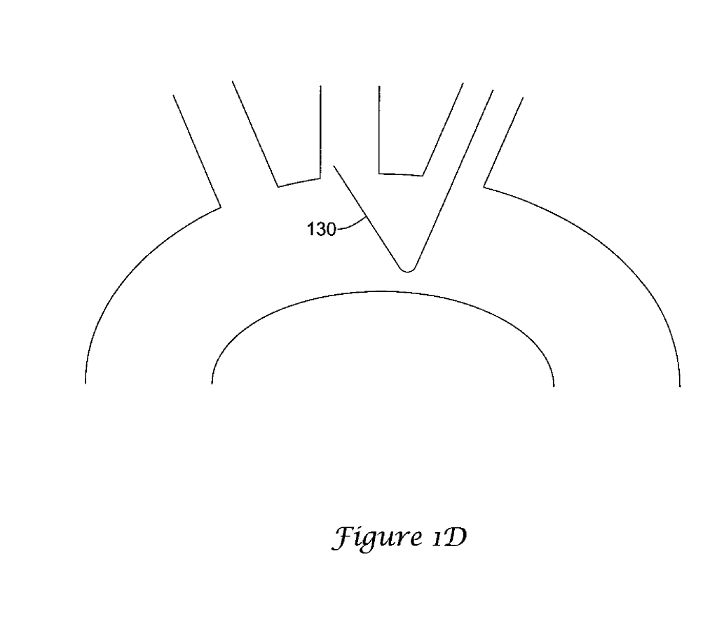

FIGS. 1A-1D, illustrate schematically the operation of the disclosed medical device delivery apparatus to deliver, for example, a guidewire within a blood vessel along a somewhat reflexive path which would otherwise be difficult to accomplish. The apparatus comprises a generally tubular element 110 which is advanced to the deployment site at the distal end of an elongated support member (not shown). Once the generally tubular element 110 is positioned, a directing element 120 (shown partially extended in FIG. 1A), initially contained within a lumen of the generally tubular element 110 in a straightened first configuration is extended from a lumen associated with the generally tubular element 110 and is caused or allowed to assume a curved second configuration as shown in FIG. 1B. A medical device, in this instance guidewire 130, having greater flexibility near its distal end than the flexibility associated with the deployed directing element 120 is then advanced from a lumen of the generally tubular element 110 (FIG. 1B) until it encounters and is deflected by the directing element 120 as shown in FIG. 1C. The internal angle associated with the deflection between the initial longitudinal axis of the generally tubular element 110 and the deflected path assumed by the medical device following deflection is typically less than 120 degrees and may be less than 90 degrees or even less than 30 degrees. Once the medical device has been redirected, it may be advanced within the vessel or other lumen to which it has been redirected as desired and any associated medical device may be deployed therein. The medical device delivery apparatus may be left in place or may be withdrawn. It will be appreciated that in the case of the guidewire 130 of FIGS. 1B-1D, once the medical device delivery apparatus 110, 120 has been withdrawn, other medical devices such as catheters, stents, and the like may be advanced over the guidewire to be positioned within the difficult to access lumen to be treated.

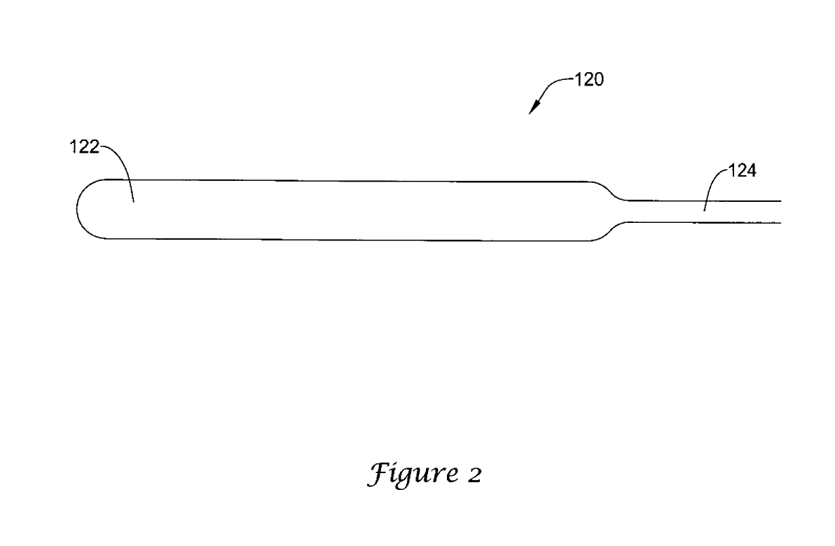

FIG. 2 illustrates an exemplary directing element 120 comprising a blade portion 122 and an advancing member 124. The use of a blade portion 122 provides greater flexibility in the curved portion of the directing element 120 as well as a broader deflecting surface which tends to ensure that the medical device to be delivered remains disposed along the deflection surface. In some embodiments, the blade portion 122 of directing element 120 may extend substantially the entire length of the medical device delivery apparatus and be available for manipulation at the proximal end of the apparatus. In other embodiments, the blade portion 122 of directing element 120 may be present only near the distal end of the directing element and attached to a proximal shaft portion 124. A stiff proximal shaft portion 124 may impart improved pushability to the directing element 120. If present, the shaft portion 124 may be formed from the same material as the blade portion 122 or it may be formed from a different material. The blade portion 122 and/or the shaft portion 124 of the directing element 120 may be made from a biocompatible material such as a polymer or metal. In some embodiments, the blade portion 122 and/or the shaft portion 124 of the directing element 120 may be made from a shape memory material. In other embodiments, the blade portion 122 of the directing element 120 may be pre-formed to assume a desired second configuration upon elastic recovery following ejection from the generally tubular element 110. As will be discussed herein, various means may be used to cause the blade portion 122 of the directing element 120 to assume the desired second configuration following ejection from the generally tubular element 110.

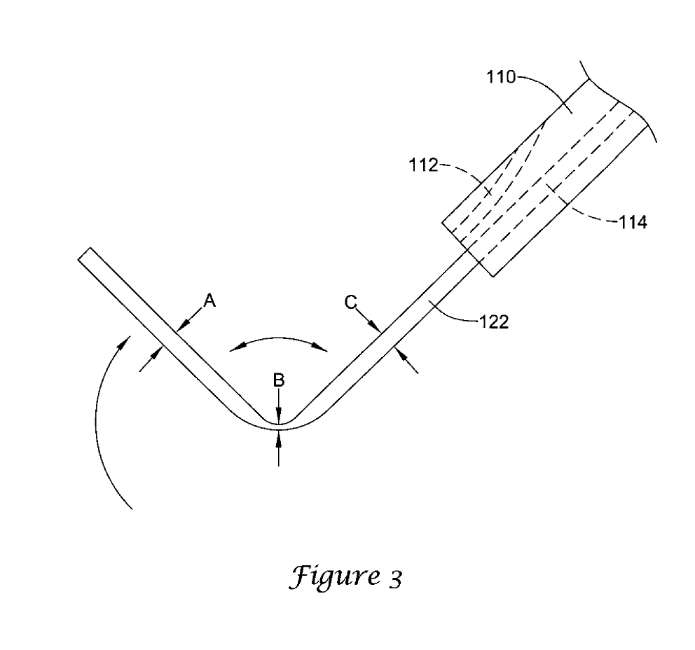

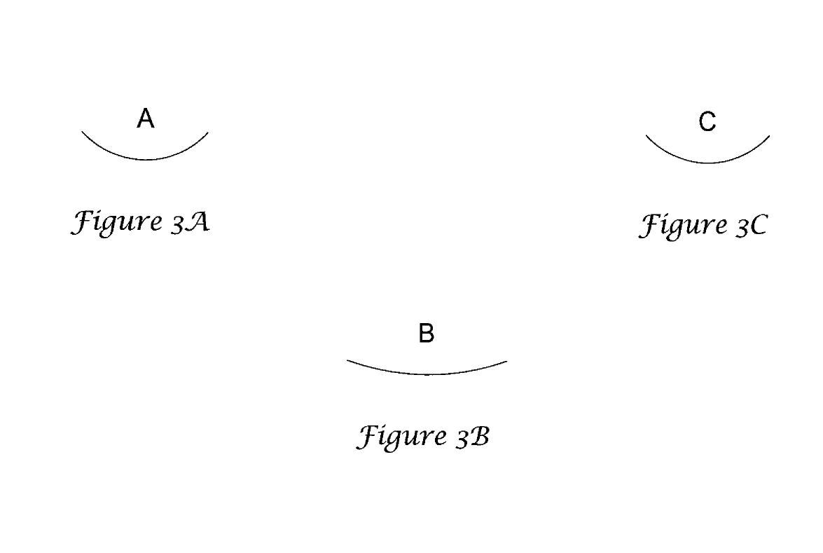

For example, as illustrated in FIGS. 3-3C, the blade portion 122 of the directing element 120 may include transverse curvature in cross-sections 3A and 3C which tends to impart a degree of flexural stiffness while the cross-section of an intermediate region (FIG. 3B) is somewhat flatter and thus likely to provide a preferred bending region and bending direction. This tendency may be enhanced, for example, by employing a shape memory material for at least a portion of the blade portion 122 such that upon warming within the body upon delivery, the blade flexes in the flattened region and assumes the configuration of FIG. 3 in which the angle indicated by the double-headed arrow is dictated by the thermal training and matches the angle expected within the anatomy at the deployment site. In some embodiments, the blade portion 122 may be designed to be relatively rigid particularly in the intermediate region to provide resistance to deflection as the medical device is advanced to push against the blade portion 122. In other embodiments, it may be desirable to maintain a degree of flexibility to allow the angle to be increased under the application of pressure by the medical device 130 to better direct the medical device toward the target.

As illustrated in FIG. 3, the generally tubular element 110 may include one or more lumens 112, 114 which are adapted to contain at least a portion of directing element 120 and/or the medical device 130. In some embodiments, the lumens may be separate, while in other embodiments the lumens may be shared by the elements to be advanced relative to the generally tubular element 110. The lumens need not necessarily extend the entire length of the apparatus or even the entire length of the generally tubular element 110 of the medical device delivery apparatus. For example, lumen 112 of FIG. 3 is illustrated as an over-the-wire lumen through which a guidewire 130 (not shown in FIG. 3 may be advanced to encounter blade portion 122.

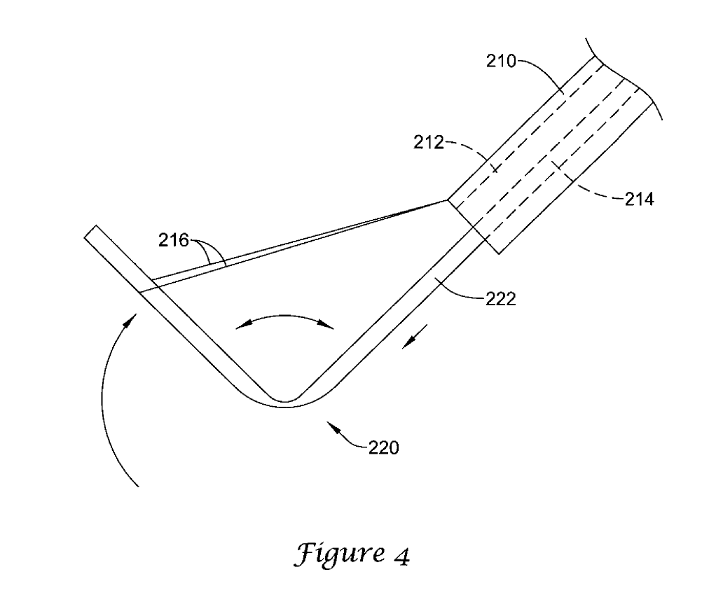

In the embodiment of FIG. 4, tethers 216, optionally initially positioned within a distal lumen of generally tubular element 210, become tensioned as the blade portion 222 is advanced and cause the blade portion 222 to buckle as illustrated. In this embodiment, the angle indicated by the double-headed arrow may be adjusted by controlling the degree of advancement of the blade portion 222 of the directing element 220. In such embodiments, the internal angle between the axis of the generally tubular element 210 and the path of the deflected medical device is typically less than 120 degrees and may be less than 90 degrees or even less than 30 degrees.

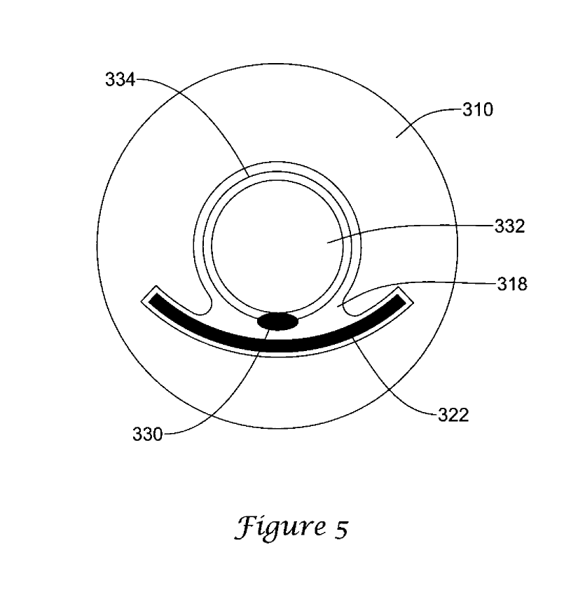

As illustrated in FIG. 4, lumen 212 is large enough to at least partially contain a medical device such as an embolic filter, a stent, the balloon portion of a balloon catheter, and the like. Although the lumens 212 and 214 are illustrated as separate, as noted above, they may be joined to form a single lumen 318 of generally tubular element 310 as illustrated in FIG. 5. Lumen 318 slidably receives both the blade portion 322 of the directing element and an embolic filter 332 attached to guidewire 330. As illustrated, the medical device, embolic filter 332 includes a removable containment element 334 which maintains the embolic filter 332 in a collapsed configuration until it has been advanced beyond the deployed directing element and into a desired location within the anatomy whereupon it may be removed to deploy the medical device, embolic filter 332. In some embodiments, the joined lumens provide sufficient lateral direction control to ensure that the medical device remains substantially centered relative to the directing element.

In other embodiments, additional blade portion structures may be desirable. For example, the blade portion of a directing element may initially be substantially flat in a first configuration and continuously longitudinally curved in a second deployed configuration. In such embodiments, the internal angle between the axis of the generally tubular element 510 and the path of the deflected medical device is typically less than 120 degrees and may be less than 90 degrees or even less than 30 degrees. In some such configurations the simple blade of FIGS. 2 and 3 may lack at least some of the self-centering capability of a laterally curved blade portion. Additionally some combinations of directing elements and medical devices may not be well suited to lateral steering by the simple transverse curvature of FIGS. 3 alone.

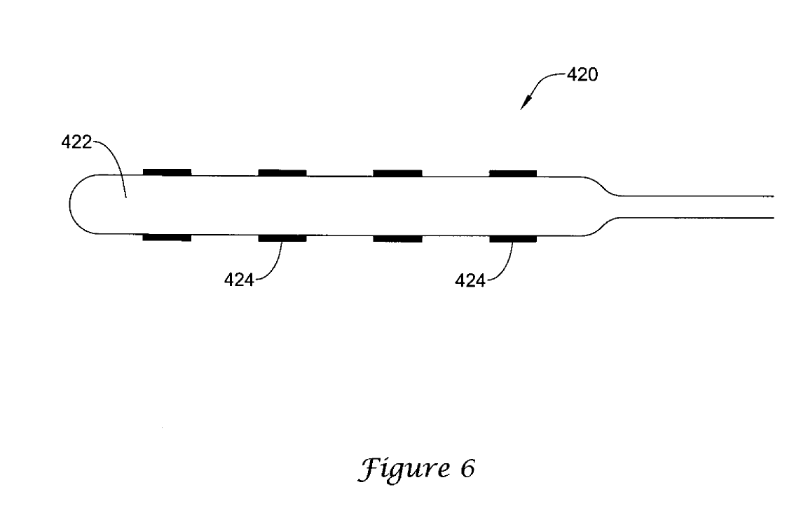

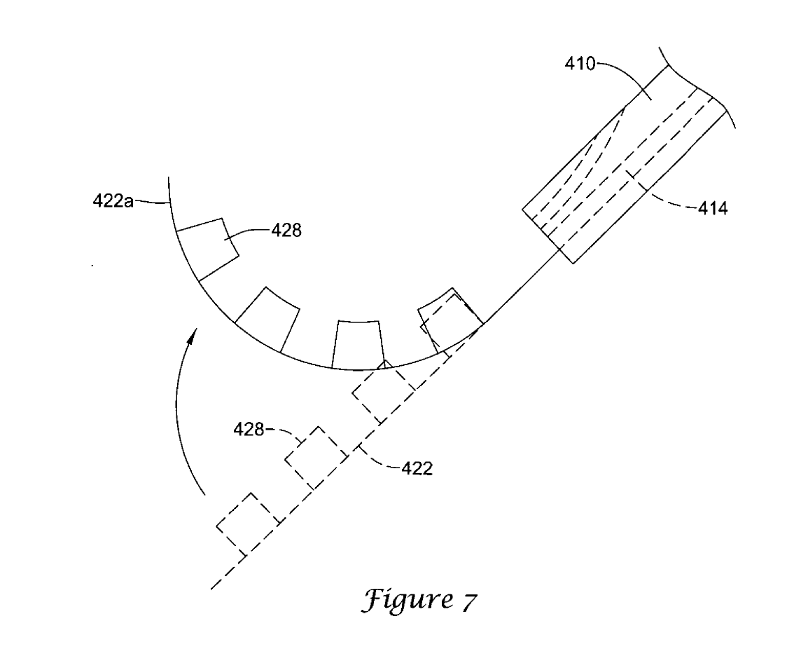

In such embodiments, one of a variety of guiding structures may be desirable. In FIGS. 6 and 7, the guiding structures take the form of edge guides 428 which constrain the medical device (not shown) to track along the blade portion 422 of the directing element 420. At the same time, the edge guides 428 need not impose unnecessary constraints on the necessary bending of blade portion 422 of the directing element as may be seen in FIG. 7. In some embodiments, the blade 422 and the edge guides 428 may be substantially coplanar in a first delivery configuration and the edge guides 428 may only assume the illustrated positions following the extension of blade 422 from the generally tubular element 410, while in other embodiments the lumen 414 may be sized to accommodate the edge guides 428 in a deployed configuration. Accordingly, in some embodiments, the blade portion 422 of the directing element may be extended to a position distal of the generally tubular element 410 with the edge guides 428 already positioned as illustrated in phantom and in other embodiments, the edge elements 428 may be substantially coplanar with the blade portion 422 of the directing element 420 until or even after the blade portion 422 has been actuated to the curved second configuration illustrated as 422A.

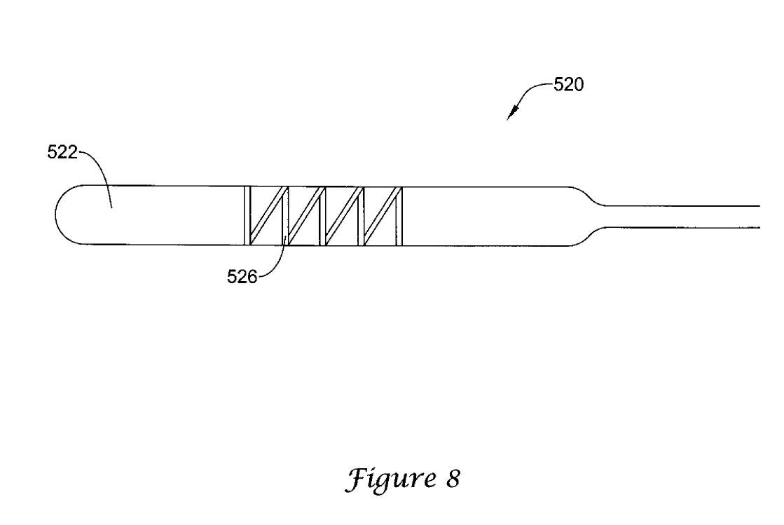

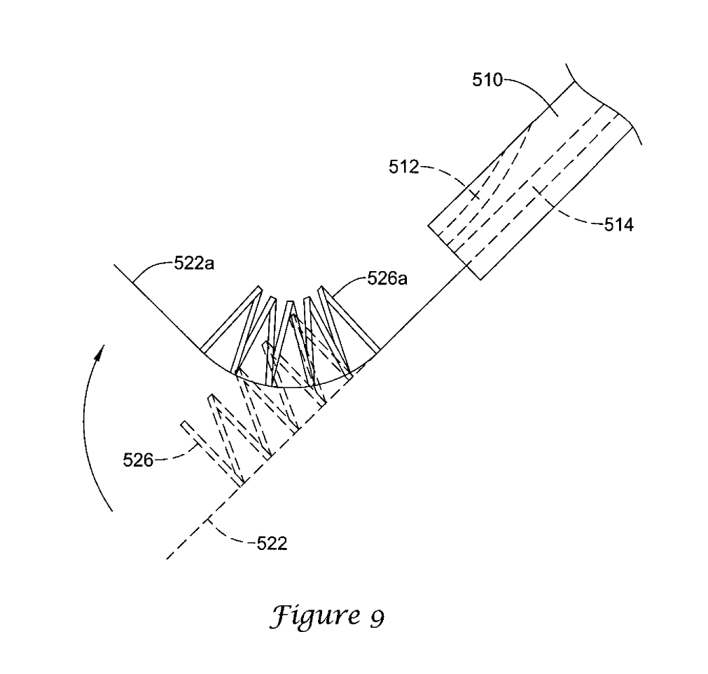

In yet other embodiments, the guiding structure may comprise a generally tubular segment of the directing element as shown in FIGS. 8 and 9. In such embodiments, the tubular segment may take the form of a coil or coils 526 and may also serve to provide a degree of control over which portions of blade portion 522 bends under the influence of the various actuating mechanisms such as elastic recovery, tethers, shape memory components, and the like. For example, the coil 526 may be pre-shaped to assume the second configuration of FIG. 9 upon ejection from generally tubular element 510 and thus to bend the blade portion 522 as indicated by reference numerals 526A and 522A. In other embodiments, the coil 522 and/or blade portion 522 of directing element 520 may be formed from shape memory materials and may flex to assume the shape indicated by the double-headed arrow upon warming to an appropriate temperature. In such embodiments, the internal angle between the axis of the generally tubular element 510 and the path of the deflected medical device is typically less than 120 degrees and may be less than 90 degrees or even less than 30 degrees.

In yet other embodiments, deflection in the region spanned by the coil may introduced by tethers resembling those of FIG. 4. In other embodiments (not illustrated) the directing element may comprise one or more tubular segments and adjacent flexible blade portions such that the blade portions flex under the influence of actuating mechanisms disclosed herein, as well as others known in the art, while the tubular portions remain substantially undeformed and provide the directing function.

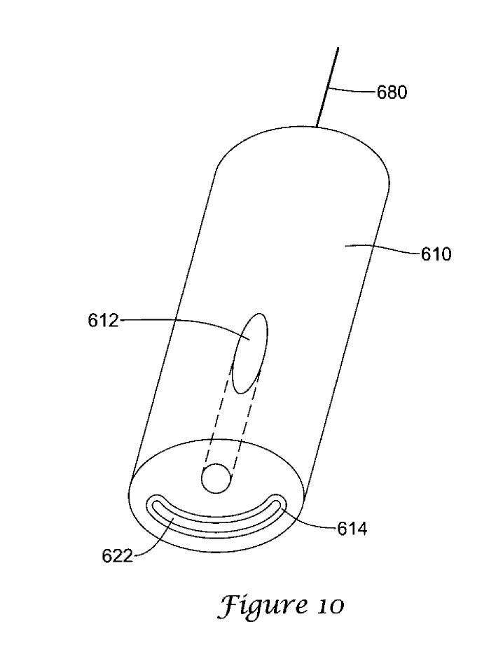

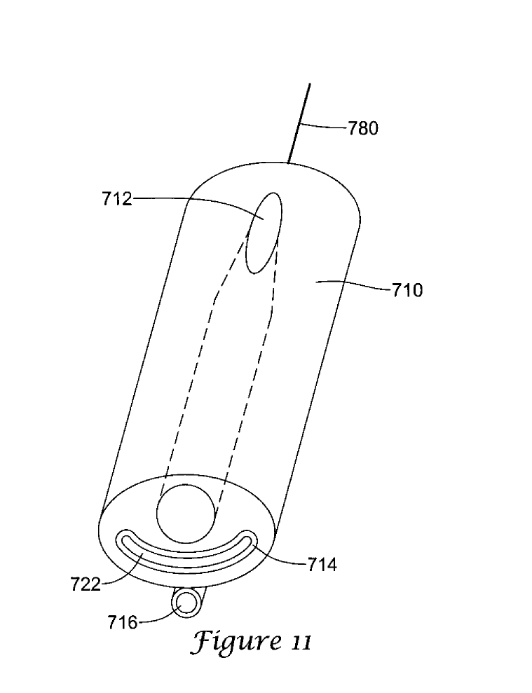

As noted earlier, in some embodiments the generally tubular element of the medical device delivery apparatus may be the distal region of a catheter which extends to a position external to the body being treated. In other embodiments the generally tubular element may be mounted on a distal region of a guidewire or hypotube as illustrated in FIGS. 10 and 11. In the embodiment of FIG. 10, the generally tubular element 610 is fixedly attached to the distal end of an elongated support member 680 which serves to advance the generally tubular element 610 through the vasculature to the deployment site. In addition, the elongated support member 680 may carry, internally or deployed alongside, advancing member 624 which is not visible in the figure. In some such embodiments, the apparatus may be advanced over a guidewire (not shown) which passes through lumen 612. When this arrangement is employed, the apparatus may be advanced over the medical device to be deflected to reach a site which has proven to be difficult to traverse by conventional means. Once positioned near the desired deflection point, the blade portion 622 of the directing element may be advanced by advancing member 624 to a position distal of generally tubular element 610 thereby providing a deflecting surface for the guidewire over which the apparatus had been advanced. Once the guidewire has been deflected and further advanced within the vasculature, the blade portion 622 may be withdrawn into the generally tubular element 610 and the generally tubular element may be withdrawn from the patient if desired.

In the embodiment of FIG. 11, the generally tubular element 710 is also mounted on a distal region of a guidewire or hypotube as illustrated in FIGS. 10 and 11. In the embodiment of FIG. 11, the generally tubular element 710 is fixedly attached to the distal end of an elongated support member 780 which serves to advance the generally tubular element 710 through the vasculature to the deployment site. In addition, the elongated support member 780 may carry, internally or deployed alongside, advancing member 724 which is not visible in the figure. In some such embodiments, the apparatus may be advanced over a guidewire (not shown) which passes through optional lumen 716 and optionally carries a medical device to be deflected such an embolic filter, stent, balloon, or the like (not shown for clarity). The medical device to be deflected may be contained within lumen 712. Although the lumen 712 is depicted as a separate lumen and is depicted as having an over-the-wire configuration it will be appreciated that lumen 712 may be coaxial with the generally tubular element 710 and/or may be joined therewith lumen 714 as a single lumen such as that of lumen 318 of FIG. 5 if desired. It will also be appreciated that lumen 612 of FIG. 10 and/or lumen 716 of FIG. 11 are capable of receiving an elongated support member associated with any of the embodiments described herein if it is desirable to introduce and deflect more than one medical device during a procedure.

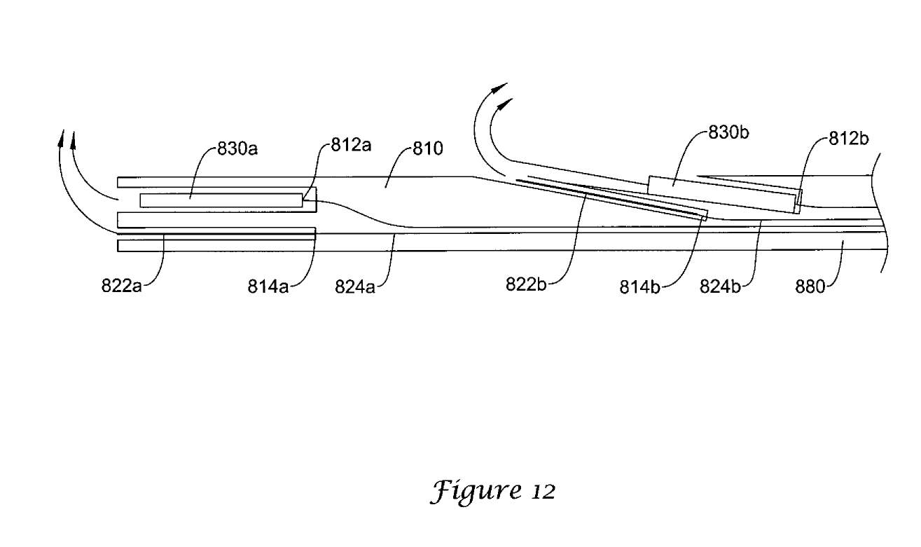

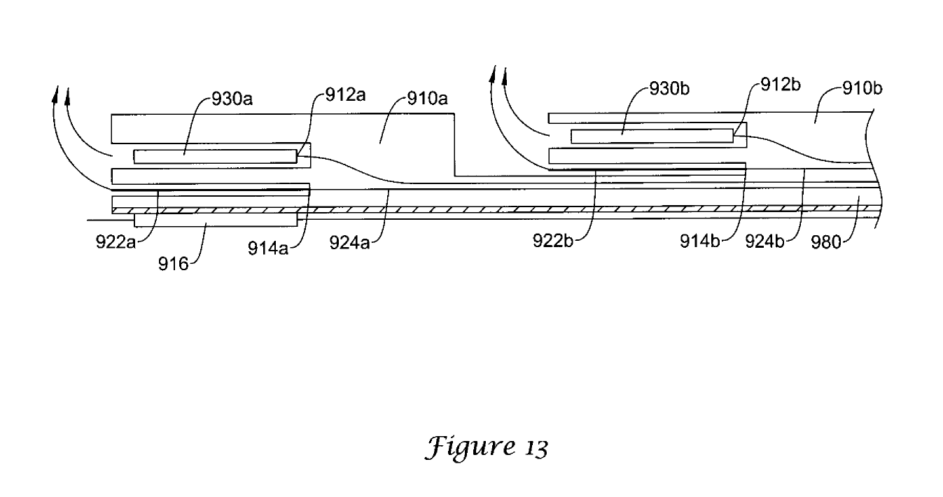

The embodiments of FIGS. 12 and 13 illustrate two additional medical device delivery arrangements which employ the principle of operation of this disclosure to direct two, or more, medical devices along paths which form an internal angle of less than 120 degrees. In the embodiment of FIG. 12, the elongated support member is a catheter 880 having a distal region 810 which includes first and second directing elements 822A, 822B disposed in lumens 814A, 814B which, when disposed distal of their respective 814A, 814B are adapted to direct medical devices 830A and 830B along the paths generally indicated by the respective arrows when the respective advancing members 824A and 824B have been advanced to position the blade portions 822A and 822B in their second configurations as indicated by the arrows and the medical devices 830A and 830B have been advanced to contact and be deflected by the second configurations of the blade portions 822A and 822B of the deflecting elements as illustrated in FIGS. 1A-1D. The deployments may occur sequentially in any order or may occur simultaneously. It will be apparent that the medical devices 830A and 830B may be the same or different and may be deployed sequentially in any order or may be deployed simultaneously. For example, medical devices 830A and 830B may both be embolic filters to be deployed within separate branches of the vasculature thereby allowing two embolic filters to be positioned by a single delivery catheter and further to allow the embolic filters to be placed by an access route which would otherwise be difficult or impossible given the angular relationship between the access route and the two branches to be protected by the embolic filters.

As illustrated, the lumens 812B and 814B are angled within the generally cylindrical element 810 to facilitate delivery along more acute internal angles than might otherwise be readily available. It will be appreciated that the lumens 812A, 812B, 814A, and 814B may be angled and or curved with respect to the longitudinal axis of the generally cylindrical element 810 of catheter 880 as desired. Similar curved and/or angled lumens may be provided in any of the embodiments of this disclosure.

FIG. 13 illustrates a variation of the apparatus of FIG. 12 in which the elongated support member 980 includes a stiffening member at least in the distal region and a portion of the generally tubular element has been removed to provide clearer exit paths. This arrangement effectively creates two generally tubular elements 910A and 910B which may be delivered together and operated independently generally as discussed with regard to the similarly numbered elements of FIG. 12 and/or the two separate generally tubular elements 610, 710 of FIGS. 10 and 11. The embodiment of FIG. 13 is illustrated as including an optional over-the-wire lumen 916 for a guidewire over which the apparatus may be advanced to the deployment site as discussed herein with regard to other embodiments.

The operation of each of the embodiments of the disclosure is similar. Following intravascular delivery of the apparatus to a deployment site, the directing element is advanced from a lumen of the generally tubular element by advancing a proximal portion thereof, typically an advancing member until at least a portion of a blade portion of the directing element having a first configuration is deployed distal of the distal end of the generally tubular element whereupon the deployed blade portion of the directing element either assumes or is caused to assumed a second configuration in which at least a portion of the deployed blade is curved to form an internal angle of less than 120 degrees with the longitudinal axis of the generally tubular element. Once the blade portion is positioned, a medical device in the same or adjacent lumen may be advanced along the directing element which causes the medical device to follow a curved path such that the distal portion of the medical device travels generally parallel to the distal portion of the directing element. Further advancement of the medical device causes the medical device to enter and/or be positioned within the lumen of a vessel. In some embodiments, the distal end of the directing element may also ender the target vessel during deployment. Once the medical device is positioned, the apparatus may remain in place, where it may direct additional medical devices, or the apparatus may be partially or completely withdrawn from the body. A similar sequence of operations may be employed for each medical device and/or each generally tubular element of a multi-tubular element apparatus.

In other embodiments, a single directing element may be used to deploy more than one medical device from a generally tubular element. In such embodiments, the first and second medical devices may be disposed within a single lumen of the apparatus or the medical devices may be disposed within separate lumens of the generally tubular element. For example, a first embolic filter may be deployed from a first lumen to be directed toward a first direction and following repositioning of the directing element, a second filter may be deployed from a second lumen.

Various modifications and alterations of this invention will become apparent to those skilled in the art without departing from the scope and principles of this invention, and it should be understood that this invention is not to be unduly limited to the illustrative embodiments set forth hereinabove. All publications and patents are herein incorporated by reference to the same extent as if each individual publication or patent was specifically and individually indicated to be incorporated by reference.

What is claimed is:

1. A medical device delivery apparatus comprising:
an elongated support member having a proximal end, a distal end, and a longitudinal axis;
a generally tubular element having at least a first lumen therethrough disposed at the distal end of the elongated support member; and
a first directing element disposed at least partially within the generally tubular element and slidable within the first lumen thereof,
wherein the first directing element has a first configuration which is generally straight and contained within the first lumen of the generally tubular element and a second configuration in which the first directing element is curved and at least partially extended distal of the generally tubular element,
further wherein the first directing element is adapted to receive and redirect a first medical device advanced along a first path generally parallel to the longitudinal axis of the elongated support member proximate the distal end thereof and thereby direct at least a portion of the medical device along a second path forming a first internal angle of less than 120 degrees with the longitudinal axis when the first directing element is in the second configuration and the first medical device is extended distal of the generally tubular element.

2. The medical device delivery apparatus of claim 1, wherein the first directing element includes at least one non-tubular region distal of the generally tubular element when the first directing element is in the second configuration.

3. The medical device delivery apparatus of claim 1, wherein the first directing element includes at least one tubular region distal of the generally tubular element when the first directing element is in the second configuration.

4. The medical device delivery apparatus of any of claims 1, further comprising a second lumen within the generally tubular element and a second directing element disposed at least partially within the second lumen of the generally tubular element and slidable within the second lumen thereof wherein the second directing element has a first configuration which is generally straight and contained within the second lumen of the generally tubular element and a second configuration in which the second directing element is curved and at least partially extended distal of the generally tubular element,
further wherein the second directing element is adapted to receive and redirect a second medical device advanced along a path generally parallel to the longitudinal axis of the elongated support member proximate the distal end thereof and thereby direct at least a portion of the second medical device along a second path forming a second internal angle of less than 120 degrees with the longitudinal axis when the second directing element is in the second configuration and the second medical device is extended from of the generally tubular element.

5. The medical device delivery apparatus of claim 4, wherein the first directing element includes at least one non-tubular region distal of the generally tubular element when the first directing element is in the second configuration.

6. The medical device delivery apparatus of claim 4, wherein the first directing element includes at least one tubular region distal of the generally tubular element when the first directing element is in the second configuration.

7. The medical device delivery apparatus of any of claims 4, wherein the first internal angle is equal to the second internal angle.

8. The medical device delivery apparatus of claim 2, wherein the first internal angle is not equal to the second internal angle.

9. The medical device delivery apparatus of any of claims 1, wherein at least one of the first directing element and the second directing element is an elongated strip.

10. The medical device delivery apparatus of claim 9, wherein the elongated strip includes one or more edge guides along each edge of the elongated strip.

11. The medical device delivery apparatus of any of claims 9, wherein the elongated strip further includes a generally tubular region comprising one of a flexible coil or braid.

12. The medical device delivery apparatus of any of claims 1, wherein the first directing element further comprises one or more tethers adapted to fix an interior angle between the longitudinal axis of the elongated support member and the distal portion of the first directing element when the first directing element is in the second configuration.

13. The medical device delivery apparatus of any of claims 4, wherein the second directing element further comprises one or more tethers adapted to fix an interior angle between the longitudinal axis of the elongated support member and the distal portion of the second directing element when the second directing element is in the second configuration.

14. The medical device delivery apparatus of claim 1, wherein the first internal angle is less than 90 degrees.

15. The medical device delivery apparatus of claim 4, wherein the second internal angle is less than 90 degrees.

16. The medical delivery device of any of claim 1, wherein the generally tubular element includes at least one lumen adapted to at least partially contain the first medical device.

17. The medical delivery device of claim 16, wherein the first medical device is a guidewire.

18. The medical delivery device of claim 16, wherein the first medical device is an embolic filter.

19. The medical delivery device of claim 18, wherein the first medical device further comprises a removable containment element.

20. The medical delivery device of claim 16, wherein the first medical device is a stent.
